(19) United States
(12) Patent Application Publication
(10) Pub. No.: US 2023/0058011 A1
(43) Pub. Date: Feb. 23, 2023

(54) METHOD APPARATUS AND SYSTEM OF WEARABLE SYNCHRONIZED MULTIPLE VITAL HEALTH SENSORS AND DATA PROCESSING AND APPLICATIONS

(71) Applicant: Bayland Scientific Inc., Pleasanton, CA (US)

(72) Inventors: Xiaosong Du, San Ramon, CA (US); Yi Lin, San Ramon, CA (US); Yuxin Zhou, Fremont, CA (US)

(21) Appl. No.: 17/820,184

(22) Filed: Aug. 16, 2022

Related U.S. Application Data
(63) Continuation of application No. 63/234,202, filed on Aug. 17, 2021.

(51) Int. Cl. A61B 5/00; A61B 5/0205; G16H 10/60

(52) U.S. Cl. CPC A61B 5/68335; A61B 5/0205; A61B 5/7282; A61B 5/7246; G16H 10/60; A61B 2562/06

(57) ABSTRACT

Apparatus and method are provided for synchronized multiple vital health measurements. In one novel aspect, an integrated wearable device with multiple sensors that can collect multiple vital health signals, digitize them, send them through wireless network to a receiver. In one embodiment, the wearable device has a plurality of different types of sensors including at least one or more acoustic-to-electric sensors collecting phonocardiogram (PCG) electrical signal and one or more electrocardiogram (ECG) sensors, a control module includes a synchronization circuitry that synchronizes measurements of the plurality of different types of sensors. In another novel aspect, a system performs a synchronized measurement using a plurality type of health-monitoring sensors, performs a correlation analysis of the plurality of measurement results using selected one or more analytical rules, and obtains a set of parameters with recognized medical values and generating one or more medical health records based on the correlation analysis.

### CROSS REFERENCE TO RELATED APPLICATIONS

This application claims priority under 35 U.S.C. § 119 from U.S. Provisional Application No. 63/234,202 entitled “METHOD APPARATUS AND SYSTEM OF WEARABLE SYNCHRONIZED MULTIPLE VITAL HEALTH SENSORS AND DATA PROCESSING AND APPLICATIONS,” filed on Aug. 17, 2021, the subject matter of which is incorporated herein by reference.

### TECHNICAL FIELD

The present invention relates generally relate to wearable apparatus and system, and more particularly, wearable vital health signals continuously sensing, data processing and applications.

### BACKGROUND

It is important for the healthcare professionals to collect multiple vital health signals, such as body temperature, electrocardiogram (ECG), heart rate, photoplethysmography (PPG), blood pressure. The traditional ways are performed by multiple medical instruments inside hospital. However, when a patient is outside of the hospital, it becomes very difficult to collect these signals in a timely manner. Although multiple measurement devices (such as thermometers, oximeters, and blood pressure monitors) are available to consumers in recent years, the user must record the respective measurements of each individual device. This proves to be a tedious routine many are reluctant to do. To approach this issue, wearable devices are introduced to collect health data, such as Fitbit and Apple Watch. However, these measurements are singular points of measurement without context or are collected over a short period of time at best. For instance, the Apple Watch with ECG capability can only record up to 30 seconds of continuous ECG data. During the data-collection time frame, the user must wear the watch on the wrist and use their other hand to rest on the watch dial for the measurement to be taken. Necessitating both hands in performing the measurement makes the process impractical and unsustainable for extended periods of time. Such procedure can only be applied to the general clinic application with only hands full of patients. When there are millions of users start to upload their daily vital health data to the server, the process has to be automated. Long-time and continuous monitoring has proven to be very valuable for catching early symptoms of arrhythmia and other heart problems.

During the COVID-19 pandemic, most of the hospitals had to close their normal operations and reserve space specifically to care for COVID-19 patients. Patients with chronic diseases had to stay home and care for themselves. Even people with perfect health were quarantined within their homes and worried about their health. During the crisis, many healthcare professionals turned to telemedicine to remotely interact with and care for patients. Although high-speed Internet and online meeting software like Zoom greatly facilitated this effort, it soon became apparent that remote communication with patients through video was not enough. The lack of vital health data from the patient's end made it very difficult for healthcare professionals to make thoroughly informed decision, compromising overall treatment effectiveness.

One of the many things that we have learned from the COVID-19 pandemic was the imminent need to extend medical capabilities to the patient's home. During the crisis, healthy, chronically ill, and COVID-19 afflicted individuals were all at home self-quarantining and monitoring their health conditions. To take care of such individuals in the most efficient and effective method necessitates wearable multi-sensor patches equipped with remote sensing capabilities. Meanwhile, due to the large user volume, there arises a need for automatic archival and analysis of the data to inform the user (and, if necessary, healthcare professionals) of any dangers indicated by the data.

### SUMMARY

Apparatus and method are for wearable synchronized multiple vital health sensors and data processing and application. In one novel aspect, an integrated wearable device with multiple sensors that can collect multiple vital health signals, digitize them, send them through wireless network to a receiver, such as a smart phone, or other mobile devices, a PC, or cloud-based data center. In one embodiment, the wearable device has a wearable size center container that can be attached to a body, a plurality of different types of sensors attached to the center container collecting a plurality sets of health signals, wherein the sensors include at least one or more acoustic-to-electric sensors collecting phonocardiogram (PCG) electrical signal and one or more electrocardiogram (ECG) sensors, a control unit mounted in the center container, wherein the control module includes a synchronization circuitry that synchronizes measurements of the plurality of different types of sensors mounted on the center container, a wearable size patch with one or more electrodes, wherein each electrode is connected to the center container. In one embodiment, the different types of sensors further comprising: a photoplethysmography (PPG) sensor, a body temperature sensor, and an orientation and motion sensor. In another embodiment, one or more assistant sensors attached to the center container, comprising one or more environmental temperature sensor, and an environmental noise level sensor. In one embodiment, the wearable device a configurable push button connected to the control module, wherein the push button is configured to perform a plurality of functions comprising an activation button, a panic button, and an event reminder button. In another embodiment, each electrode is connected to the center container with a button-sized metal connector. In yet another embodiment, the wearable device further includes a wireless communication circuitry, wherein the wireless communication circuitry communicates with one or more smart devices through a wireless network. In one embodiment, the wearable device is configured to monitor multiple vital health signals continuously and wirelessly. In other embodiments, the wearable device is used in one of different applications comprising in a telemedicine application, in a quarantined environment, within a hospital, and in an operation room. The wearable device is attached to a user to continuously monitor a respiration sound caused by COVID-19 or other lung disease based on synchronized ECG and PCG obtained. In yet another embodiment, attached to a patient to continuously monitor a heart performance during a pacemaker operation, and wherein the heart performance is determined based on synchronized ECG and PCG obtained. In one embodiment, the wearable device further includes one or more convertors connecting to the one or more micro acoustic-to-electric sensors and the array of voltage electrodes, wherein one or more convertors digitizes sensor waveform outputs, a non-volatile storage that stores the digitized waveforms, and a user interface unit that receives one or more user configurations for the apparatus.

In another novel aspect, a system performs a synchronized measurement using a plurality type of health-monitoring sensors including at least one or more acoustic-to-electric sensors collecting phonocardiogram (PCG) electrical signal and one or more electrocardiogram (ECG) sensors, obtains a plurality of measurement results from the synchronized measurement, wherein the plurality of measurement results includes different types of measurements that are all synchronized, performs a correlation analysis of the plurality of measurement results using selected one or more analytical rules, and obtains a set of parameters with recognized medical values and generating one or more medical health records based on the correlation analysis. In one embodiment, the system also digitizes one or more waveforms obtained from corresponding sensors and analyzes digitized data with preconfigured an algorithm selecting from a wavelet, a short-time fast Fourier transformation (FFT), and a deep learning algorithm. In one embodiment, the set of parameters with recognized medical values comprising electromechanical activation time (EMAT), EMAT percentage, a left ventricular ejection fraction (LVEF), and blood pressure. In another embodiment, the EMAT is generated by correlating a set of synchronized ECG and PCG measurements obtained. In yet another embodiment, the LVEF is obtained by negatively correlating the generated EMAT. In one embodiment, the plurality of measurement results is obtained continuously and wirelessly from a wearable patch that collects the plurality of synchronized measurements. In another embodiment, blood pressure reports are generated continuously by performing a calibration, identifying and digitizing S2 of continuously obtained PCG waveforms that are synchronized with an obtained ECG. In one embodiment, the system further identifies one or more problematic sections based on the correlation analysis. In another embodiment, the system generates one or more customized reports based on the correlation analysis.

This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 illustrates exemplary diagrams of top-level apparatus and system and the mechanical design of the wearable synchronized multiple vital health sensors combined with the wearable patch.

FIG. 2 is the exemplary drawing of how to put the wearable synchronized multiple vital health sensors onto the body.

FIG. 3 is an exemplary drawing of system architecture of the wearable synchronized multiple vital health sensors.

FIG. 4 is an exemplary drawing of using the wearable synchronized multiple vital health sensors to monitor heart performance during the anesthesia procedure and operation inside a hospital.

FIG. 5 is an exemplary drawing of using the wearable synchronized multiple vital health sensors in the telemedicine application.

FIG. 6 is an exemplary drawing of using the wearable synchronized multiple vital health sensors within a hospital, especially under the quarantined environment.

FIG. 7 illustrates exemplary diagrams of extracting EMAT parameters from the synchronized ECG and PCG signals.

FIG. 8 is an exemplary report on ECG which generated by the backend AI driven analysis software.

FIG. 9 is an exemplary report using synchronized ECG and PCG which generated by the backend AI driven analysis software.

FIG. 10 is an exemplary report shown a disease pattern using synchronized ECG and PCG which generated by the backend AI driven analysis software.

FIG. 11 is an exemplary long-term report using synchronized ECG and PCG which generated by the backend AI driven analysis software.

FIG. 12 is another exemplary long-term report using synchronized ECG and PCG which generated by the backend AI driven analysis software.

FIG. 13 is an exemplary display of using the wavelet to analyze the synchronized ECG and PCG signals.

FIG. 14 is the synchronized ECG and PCG signals collected from COVID-19 patient with heavy respiration sound.

FIG. 15 is the synchronized ECG and PCG signals collected from congenial heart disease patient.

FIG. 16 is the synchronized ECG and PCG signals are used to continuously monitor the blood pressure during the anesthesia procedure.

FIG. 17 is the X-ray image capture of using the wearable synchronized multiple vital health sensors during pacemaker implant operation.

FIG. 18 is an exemplary circuit block diagram of the wearable synchronized multiple vital health sensors.

FIG. 19 illustrates an example diagram of the correlation between EMAT and LVEF.

FIG. 20 illustrates an exemplary flow chart for the system to derive/generate medical records from synchronized multiple vital health measurements in accordance with embodiment of the current invention.

### DETAILED DESCRIPTION

Reference will now be made in detail to some embodiments of the invention, examples of which are illustrated in the accompanying drawings.

The Patient Monitor (PM) is a fundamental instrument in today's hospital, especially in the ICU and operation rooms. It collects ECG, PPG, blood pressure, body temperature and other vital parameters from the patients. Most of the devices are the size of a shoe box with a screen displaying the vital parameters and multiple wires connected to the body of the patient that the vital parameters can be collected. The Patient Monitor has proven to be an essential and effective monitoring instrument in modern medical practice. However, when the patient leaves the hospital, it becomes very difficult to collect his/her vital health parameters. Although most of the Patient Monitor can be hand-carried, it is very cumbersome and uncomfortable for the patient to carry a shoebox size device with multiple wires attached to the body. When the patient connects to a Patient Monitor, it is impossible to move around the house and conduct normal daily activities. Another challenge is that most of the Patient Monitor devices need professional knowledge to operate, especially when the device detects alarm/warning signals. The COVID-19 and future pandemic had posed new challenges for patient monitoring. During the COVID-19 crisis, the medical professionals had to wear cumbersome protection gear to interact with the patients. All monitoring devices have to go through a tedious process to be thoroughly sanitized so that they can be reused on other patients. For many patients with mild symptoms of COVID-19, they have to be self-quarantined at home. Without Patient Monitor at home, the patient did not know their health situation and may miss the best medical rescue window. During the COVID-19 crisis, many healthcare professionals had to switch to telemedicine to remotely take care patients. Although high speed Internet and online meeting software, like Zoom, greatly facilitated this effort, it is very clear that remote communication with video is not enough. The lack of the vital health data from the patient makes it difficult for the healthcare professionals to make prompt decisions.

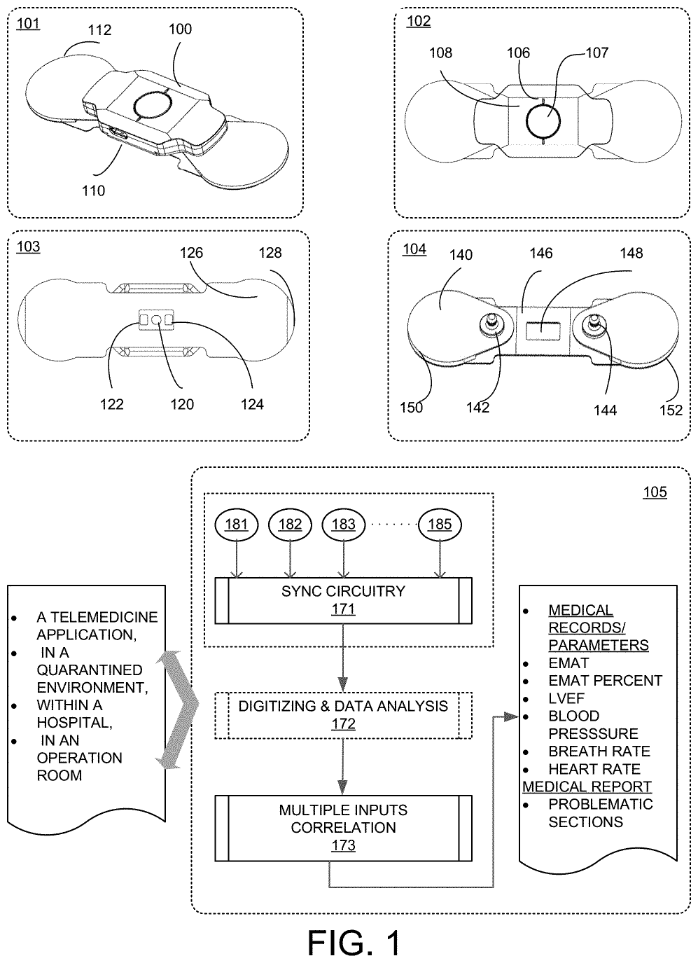

FIG. 1 illustrates exemplary diagrams of top-level apparatus and system and the mechanical design of the wearable synchronized multiple vital health sensors combined with the wearable patch. Diagram 101 illustrates an exemplary general view of the wearable device with a center container 100 and a disposable patch 112. Diagram 102 illustrates exemplary details of the center container. Diagram 103 illustrates exemplary mounting of a plurality of different types of sensors. Diagram 104 illustrates exemplary details of electrodes, center container and the disposable patch.

In one novel aspect, a wearable personal health monitoring device with multiple sensors, which can synchronously collect multiple vital parameters from the user who wears it is provided. The wearable device transmits the digitized vital health parameters through a wireless network, such as Bluetooth or Wi-Fi to the smart device, such as a smart phone, tablet, PC or other similar devices.

The wearable capability is very important. It enables the user to perform a self-administered test. Another big advantage of the wearable device compared to a handheld device is that it can perform not only short-term testing but also long-term continuous testing without discomfort. It is common sense that it is very difficult for the user to hold a device consistently for a long period of time. One example is the ECG feature of the Apple Watch. It requires the user to use the free hand that is not wearing the watch to hold the dial of the watch to perform the test. In this case, it is very difficult for the user to hold it for a long period of time.

However, designing a long-term wearable sensor involves many engineering challenges. One of the engineering challenges of a wearable device is that the device must be very small and light so that it can be worn. Although most of the electronic components nowadays are very small, it poses lots of challenges to the mechanical parts. The present invention has an acoustic sensor in it so that it can detect sound, like a micro stethoscope. As we all know, the stethoscope has a big stethoscope head as an acoustic chamber to collect sound. To design a wearable micro stethoscope, we have to get rid of the traditional large stethoscope head. A micro acoustic chamber and the whole enclosure of the wearable device are carefully designed to maximize the acoustic resonance character at a desirable frequency range to achieve high sensitivity and good noise immunity. It requires not only major innovation in sensor and acoustic mechanic design but also in circuitry and digital signal processing to achieve the desired performance.

Another challenge is to control the weight and overall dimension of the mechanic enclosure. In order to be worn on the body for a long time, the wearable sensor cannot be heavy and bulky. In general, the weight of the wearable sensor has to be less than 15 grams and the overall mechanical dimension has to be controlled within 6 cm×3 cm×1 cm. Many long-term wearable sensors use a flexible circuit and flexible enclosures, that the whole device is disposable for one-time application. However, it is not a cost-effective design. It will create lots of electronic waste in large volume applications. The present invention adopts a two-part design.

In one embodiment, the wearable device has multiple working modes. In one mode, the collected data sends to a smart device (such as a smartphone, tablet, PC or other devices) through the wireless network in real time. In another mode, the device saves the collected data in its internal storage; meanwhile, it sends the data in real time to the smart device. In yet another mode, the wearable device saves the collected data until the sampling is finished, then sends the data to the smart device. In yet another mode, the wearable device saves the collected data in its internal storage and periodically sends the current collected data through the wireless network to the smart device. When the sampling is finished, it will send the whole data set through the wireless network to the smart device. The corresponding control software (APP) in the smart device side also has multiple working modes. It can collect the data from the wearable device and relay them to the cloud-based data center in real time; or the smart device can save the data in its local storage; or the smart device can save the collected data in its local storage, meanwhile relaying them to the cloud-based data center; or the smart device can save the collected data in its local storage, and once a while, reply to the data center and when the sampling finished. It relays all the collected data to the cloud-based data center.

In one embodiment, the wearable device can be used to continuously collect data from multiple sensors in a synchronized manner. It can also be used to collect the data from the multiple sensors in a synchronized mode for a short period of time. The wearable device can be configured in a mode where when the user presses the button on the device, it immediately starts to collect data or enters a continuously collecting mode. It will automatically this action. The wearable device can be patched on various parts of the body using a clipped-on patch to monitor variable vital health information.

In one embodiment, the wearable patch has a re-usable and rechargeable center part (100) which contains all the sensors and circuits, and a disposable patch (140), which contains two electrodes (150, 152). The two parts connect together by two metal buttons (142, 144) on the patch to be snapped into the center part (100). The center part (100) can be designed in a rigid enclosure or a flexible enclosure. It has a push button at center (107), which can be used as a power button to turn on and off the sensor when the user presses the button for a long period of time. When the power is turned on, the push button (107) can be configured as multiple functions by the APP. In one application scenario, the push button (107) can be configured as a panic button. When the user presses the button, the wearable sensor will send a panic alarm to the smart device, the smart device will reply to the panic alarm to the cloud-based data center and the data center will forward the panic alarm to the smart devices of the user-designated persons, such as relatives and healthcare professionals.

In another application scenario, the push button (107) can be configured as a start data acquisition button to inform the wearable sensor to collect data. In another application scenario, the push button (107) can be configured as an event reminder during the continuous data recording. During the continuous data recording, when the user feels discomfort, they can push this button and the wearable sensor will record this event along with the data from other sensors. It will remind the data analysis algorithms that the data around the event are very sensitive and need to pay more attention. There is a motion/gyro sensor and a configurable multi-purpose button in the wearable device. The motion/gyro sensor can be used to detect and accumulate the movement of the user in order to estimate the daily movement and further derive the calorie burned each day. Meanwhile, when the motion/gyro sensor detects a sudden drop or abnormal movements, it can inform the wearable device to send an alarm/warning to the smart device and further relay the message to a group of people designated by the user (such as healthcare professionals or relatives). In case of a false alarm/warning, the user can use the smart device to cancel the alarm/warning. The configurable multi-purpose button can be configured to turn on/off the device. It can be configured that during the long term continuously monitoring when the user presses the button, it means that the user may feel discomfort or certain pre-defined possible events. The press-button actions will be recorded with samples from other sensors to help the backend AI-driven data analysis software analyze the data and they will also be shown in the report to remind the healthcare professionals that certain events happened during the data recording. In another scenario, the button can be configured as a panic button. When the user presses the button, an alarm/warning will send to the smart devices designated by the user so that the owners of the smart devices can take appropriate actions. In the applications within the hospital, the button can be configured as a calling button so that when the patient presses the button, the smart devices of the patient's healthcare professionals will get an immediate alarm/warning.

There are two LEDs (106, 108) on the center part (100). One is in blue (106), which indicates the power. The other (108) is red, which is used during charging. It will be red when starting charging. It will be turned off when the battery is fully charged. There is a micro-USB connector (110) at the side of the center part (100). It can be used to charge the battery and download internal storage data. There will be no micro-USB connector for the model of wearable sensor that the user can wear during a shower. All the charging and data download are done wirelessly.

The wearable device contains multiple sensors that can collect electrocardiogram (ECG), sound, photoplethysmography (PPG), body temperature, body orientation and motion, environment temperature, and environment noise level. From the readings of these sensors, the wearable device has the capability to monitor ECG, heart sound, heart performance, lung sound, sound generated by intestines, as well as the sound generated by the blood flow in arteries and veins, oxygen saturation, dynamic blood pressure, respiration rate, body temperature, and other vital health information. One very important capability of the wearable device is that the multiple sensors can be sampled simultaneously so that the time correlation between the vital health signals can be explored in the later analysis. In one example, on the back side of center part (100), there are three sensors. One is the acoustic sensor (120), one is the body temperature sensor (122), and one is the photoplethysmography (PPG) sensor (124). The front side of the disposable patch (140) has a center window (148) so that the sensor can touch the skin of the user. It has a peelable center part (146) to expose the adhesive underneath it. By using this adhesive, the disposable patch can closely connect to the center part (100). The user can hold the edge of the back side of the disposable patch (128) and peel off a plastic protection film (126) to expose the adhesive on the back side with two electrodes (150, 152). By using this adhesive, the patch can closely contact the skin of the user. The two metal buttons (142, 144) are connected to the two electrodes and can be snapped into the center part (100) so that the ECG signals detected by the two electrodes (150, 152) are connected to the wearable sensors. The two metal buttons also enforce the mechanic connection between the center part (100) and the disposable patch (140).

In one novel aspect, a synchronized multiple vital health measurements system is provided. The system performs a plurality type of synchronized health measurements, collects a plurality of measurement results from the synchronized measurement, wherein the plurality of measurement results includes different types of measurements that are all synchronized, performs a correlation analysis of the plurality of measurement results using selected one or more analytical rules, and a set of parameters with recognized medical values and generating one or more medical health records based on the correlation analysis. Diagram 105 illustrates exemplary components of the synchronized multiple vital health measurements system. A plurality of sensors, such as sensors 181, 182, 183, and 185, are controlled by a synchronization circuitry 171. Sensors 181, 182, 183, and 185 are of different types, including one or more ECG sensors, PCG sensors, and acoustic sensors. In one embodiment, sensors 181, 182, 183, 185 and synchronization circuitry 171 are integrated in one wearable patch. In one embodiment, the wearable patch can be configured to collect measurement data continuously and wirelessly. At step 172, the synchronized measurement results are digitized, and data analysis is performed with preconfigured algorithm. In one embodiment, the digitization and/or the data analysis is performed by a circuitry in the wearable patch. In another embodiment, the wearable patch transmits all or part of the measurement results to a receiver such as the measurement results are digitized and/or analyzed remotely. At step 173, a set of parameters with recognized medical values is obtained one or more medical health records are generated based on the correlation analysis. Medical records and/or parameters generated include EMAT, EMAT percentage, LVEF, blood pressure, breath rate, heartbeat rate and etc. Further, the medical report can be generated based on user configuration and/or comparison with historical data, such as problematic sections identified. In one embodiment, the data correlation is performed remotely on a server through the network interface. In another embodiment, the data correlation is performed locally on the wearable device/apparatus. In one embodiment, the synchronized multiple vital health measurements system is advantageous when applied to different use cases. The exemplary use cases include telemedicine application, in a quarantined environment, within a hospital, and in an operation room.

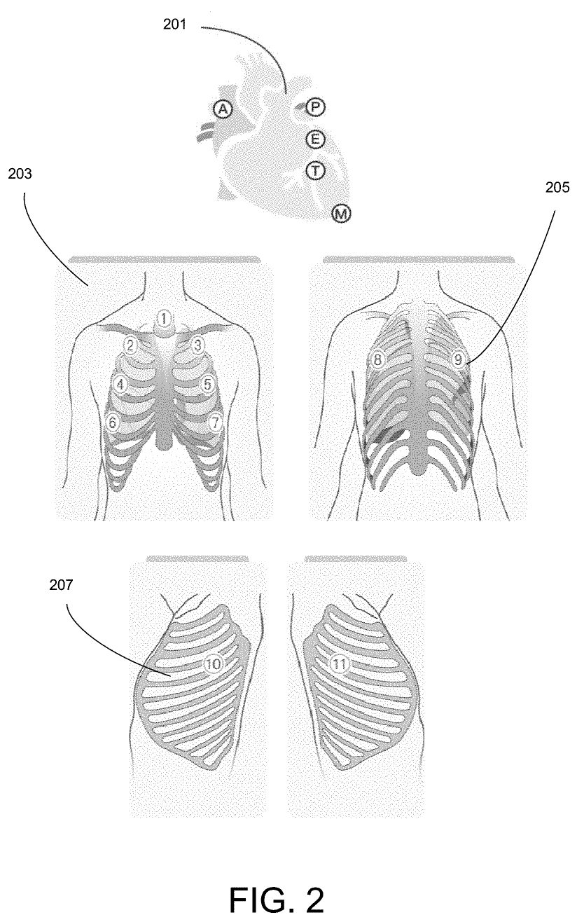

FIG. 2 has shown that the sensor can be placed on various positions (201) of the heart to detect the sound generated inside the heart. In one embodiment, the wearable patch provides measurement results of the micro stethoscope function. The wearable sensor can be placed at various positions in the front, on the back and at the side of the chest to detect the sound coming from the lung. For example, there are six positions in the front part of the chest (203), two positions on the back (205) and two positions at the side of the body. These positions are defined by the medical community from many years of research and practice. There are two important advantages of the wearable micro stethoscope compared with a traditional hand-carried stethoscope or a hand-carried digital stethoscope. When the user puts the hand-carried stethoscope onto the body, the pressure applied to the body is very important. The harder it is pressed, the greater the signal strength. Healthcare professionals can easily adjust the pressure according to their training and experience. However, it is very difficult for inexperienced patients to adjust the pressure applied to the stethoscope. Therefore, when inexperienced patients use the stethoscope, different patient may generate different results. Even for the same patient, a different tests may generate different test results, making test consistency a concern. The wearable micro stethoscope is patched onto the body without extra pressure. Therefore, test consistency is not a problem even for inexperienced patients. Another advantage of the wearable micro stethoscope is that it can perform long-term continuously testing with consistent results. It is impossible for a user, even a well-trained healthcare professional, to hold a hand-carried traditional stethoscope or hand-carried digital stethoscope steadily for a long period of time.

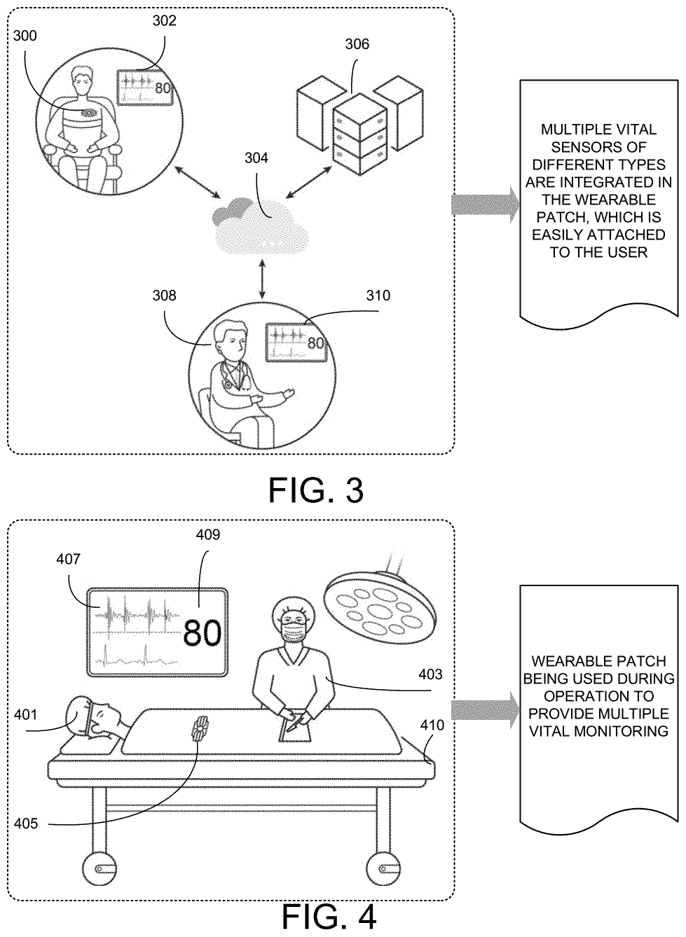

In one embodiment, the wearable device can be configured to collect measurements continuously over a long period of time, for example, over days or months. It also provides the flexibility and accuracy of being handled by users who are not medical professional. Furthermore, the measurement results, which are synchronized, can be processed dynamically and provides real time medical recognizable data/records without using cumbersome devices. It allows adapting its use in operation rooms, such as for pacemaker operations. The following diagrams in FIG. 3 to FIG. 6 illustrate exemplary use cases for the wearable device/patch.

FIG. 3 illustrates the application scenario of the wearable multiple vital health sensors. The user wears the sensor (300), it collects multiple vital health signals, including sound, ECG, PPG, body temperature, body motion, orientation and acceleration and digitizes these signals. These data can be optionally stored in the local storage of the sensor. These data can be sent to a smart device (302) (such as smart phone, tablet, computer, etc.) through a local wireless network (such as Bluetooth, ZigBee, Wi-Fi, etc.). The smart device can analyze and store the data as well as relay the data through wireless network (304) (Wi-Fi, 3G/4G/LTE/5G, etc.) to a remote data server or data server cluster (306). The server-side software can store, archive, and analyze the data. Data mining software, deep learning algorithms and other DSP algorithms (such as wavelet, short-time FFT, adaptive filtering, etc.) can be used to analyze the data. The time correlation of the data between different sensors will be explored and examined as well. A set of parameters with recognized medical values (such as heart rate, EMAT, blood pressure, breath rate, body temperature, etc.) will be extracted from the data. The received data and parameters extracted from the data will be compared against a set of known disease patterns and alarm/warning thresholds. The server-side software eventually will generate a report. If the data and parameters reach the alarm/warning thresholds or match a certain disease pattern, the alarm/warning information will be put into the report and marked with special color and font. Another important capability of the server-side software is to compare the received data against the historical data from the same user and extract the health tendency information of the user. The report will be sent back to the user and the third party that user has designated (308) (such as healthcare professionals, relatives, etc.). The third party that the user has designated can retrieve the user data (310) from the server or server cluster (306) for further analysis. In certain applications, the user, server, or server cluster and third part can be physically located at different places as far as the Internet can connect them. In other applications, the user, server, or server cluster and the third party can be physically located at the same place. For example, they are all located within an operation room or within a hospital.

FIG. 4 is an illustration of the wearable multiple vital health sensor being employed to monitor the multiple vital health signals of the patient (401) during an operation (410). The wearable device can be used within the hospital to monitor multiple vital health signals wirelessly. In one application, the wearable device is placed on the chest of the patient to monitor multiple vital health signals (especially the heart performance) during the anesthesia procedure, throughout the operation, during post-operation recovery and post-discharge recovery. The wearable sensor (405) is patched on the chest of the patient (401), multiple vital health information is collected and sent to the monitoring device (407) to display in real time. In FIG. 4, the heart sound and ECG signals are displayed, and heart rate (409) is captured from the heart sound and ECG to be displayed as well. The doctor (403) can easily get various vital health signals and critical medical indicators of the patient during the operation. The traditional Patient Monitor has to use several wires to connect to various parts of the patient's body to collect the vital health signals. These wires are cumbersome in the packed operation environment. These wires have to be cleaned, sanitized, and organized after the operation. The wearable sensor wirelessly connects to the monitoring device and the patch is disposable; the device is small and easy to be cleaned and sanitized. There are no extra wires to bother the surgeon and nurses inside the operation room. In case of malfunction of the wireless network, just one USB cable can be employed to connect the wearable sensor to the monitor device to continue to monitor. The wearability, wireless connectivity and continuous monitoring features of the wearable multiple vital health sensors are played critical roles in this application.

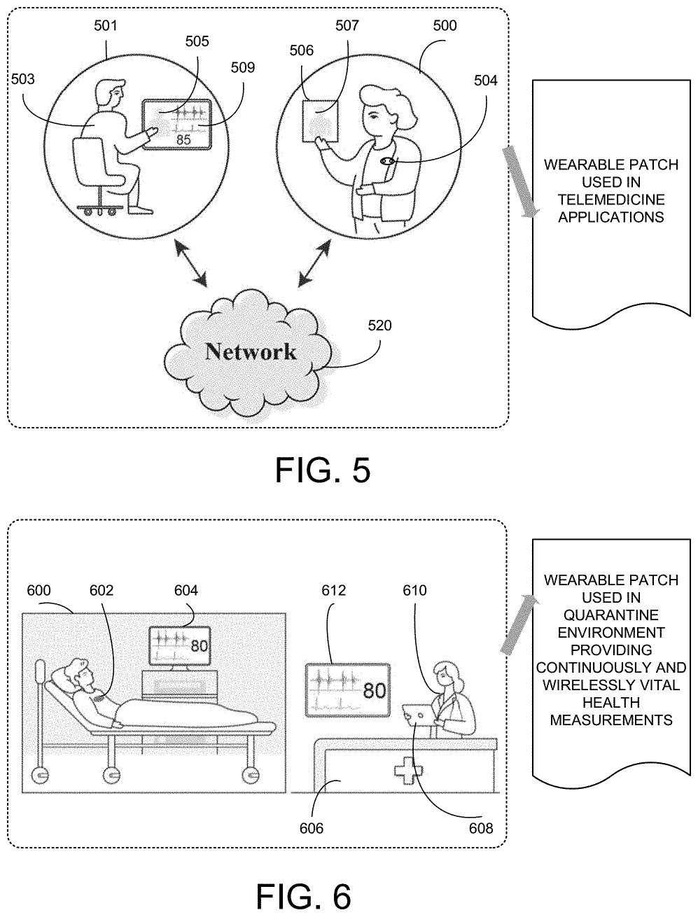

FIG. 5 illustrates the wearable multiple vital health sensors employed in a telemedicine application. The patient (500) wears the sensor (503) and has a smart device (506) (such as a smart phone, tablet, notebook, etc.). The patient can communicate with the doctor using the APP or software (507) (such as Zoom, Google meeting, etc.) installed on the smart device. The multiple vital health signals collected by the sensor (503) are sent to the smart device (506), then relay through the Internet (520) to the doctor's office (501). The collected data can be routed to a cloud-based data center and AI-based analysis algorithms will be applied and the results can be displayed on the healthcare professional's screen as well. The healthcare professional can also issue the device to the patient remotely and the patient can learn how to use the device through a demo video or online help so that he or she can use the device to collect multiple vital health signals by himself or herself. The data can be sent to healthcare professionals, and/or a cloud-based data center to automatically analyze using AI based algorithms. The analysis results can be sent to the user and his/her healthcare professionals. The doctor (503) uses a smart device (505) (such as a smart phone, tablet, notebook, etc.) to communicate with the remote patient. The doctor can not only communicate with the remote patient through audio and video but also examine the multiple vital health signals (509) in real time. This greatly improves treatment accuracy and effectiveness by streamlining the doctor and patient's communication. The patient usually lacks medical training and cannot handle sophisticated medical devices with the greatest accuracy by themselves. The wearability, easy-to-use and consistent nature of the wearable multiple vital health sensors are the key to the success of the telemedicine application.

FIG. 6 illustrates the wearable multiple vital health sensor is employed in a quarantined environment. In another application, when the patients have a contagious disease (such as COVID-19) and are quarantined, the healthcare professional can instruct the patient to put the wearable device on by letting the patient watching a short demo video demonstrating the device usage themselves. The healthcare professionals can collect the vital health signals without directly being in contact with the patient. The wearable device can also be used in a telemedicine application. The healthcare professional can teach the patient how to use the wearable through video conference software by remotely turning on the device and setting device to various working modes to collect multiple vital health signals, which can then be remotely transmitted and displayed on the healthcare professional's screen in real time. The patient with a contagious disease (such as COVID-19) stays in a quarantined room (600) and wears the wearable multiple vital health sensor (602). The sensor collects the multiple vital health signals and sends to the local smart device (604) inside the quarantined room to display and the local smart device (604) relies on the data to the server (612) at the nurse station (606). The healthcare professional (610) can monitor the vital health signals of the patient through the display of the server (612) or through a smart device (608). During the COVID-19 crisis, the healthcare professionals had to wear full protection gear to visit the quarantined room. It was difficult for them to use the traditional stethoscope to examine the patient due to the protection gear. The traditional Patient Monitor (PM) with multiple wires became cumbersome in an ICU environment. Further, it is a painstaking and time-consuming job to sanitize the PM and associated wires. A wearable device with a micro stethoscope and continuous monitoring capability greatly helps healthcare professionals perform effectively even in a quarantined environment. They can remotely monitor the multiple vital health signals, especially the sound from heart or lung. Meanwhile, it is much easier to sanitize the small wearable sensor compared to bulkier alternatives.

In one novel aspect, the synchronized measurement results are collected, analyzed, and a synchronized data-based correlation is performed. With the different types of vital health measurements being synchronized, the measurement results are used to generate/derive medical recognizable parameters/records without using the traditional cumbersome medical devices, which mostly require being operated by a medical trained professional. In one embodiment, the wearable miniature digital stethoscope is embedded into the wearable sensor so that the wearable sensor can digitize the heart sound (phonocardiograph), lung sound and sound generated by the intestines. The digitized sound signal can be acquired in a synchronized mode with ECG, PPG, and signals from other sensors. Therefore, the time correlation between different vital health signal can be explored. The time correlation between PCG and ECG can derive a parameter called electromechanical activation time (EMAT). From the clinical experiments, the EMAT is negatively correlated to the left ventricular ejection fraction (LVEF). Since the LVEF is widely used to measure the heart performance, the EMAT can also be used to measure the performance of the heart. One advantage of the wearable PCG and ECG is that the wearable device can collect PCG and ECG continuously. Therefore, it can be used to continuously monitor the heart performance. One clinical application is that during the operation of placing pacemaker, the doctor uses the wearable PCG and ECG sensor to continuously monitor the heart sound and heart performance to make sure the placement of the electrodes of the pacemaker can not only generate the proper pacing signals but also have minimum negative impact on the heart performance.

In another embodiment, the PCG and ECG signals collected by the wearable sensor can clearly identify various paraments in heart sound and ECG. In one application, using the PCG can identify various abnormal noises in congenial heart diseases. The second heart sound (S2) in the PCG signal is produced by the closing of the atrioventricular valves and semilunar valves. It is closely related to the blood coming back to the heart driven by the blood pressure. Another aspect is that the clinical experiments have proven that the wearable device can be used to continuously monitor the blood pressure. During the COVID-19 crisis, doctors used wearable PCG and ECG sensors on COVID-19 patients. They found that for moderate and severe COVID-19 patients, the heavy respiration sound that came from the lung could be observed in the PCG signal.

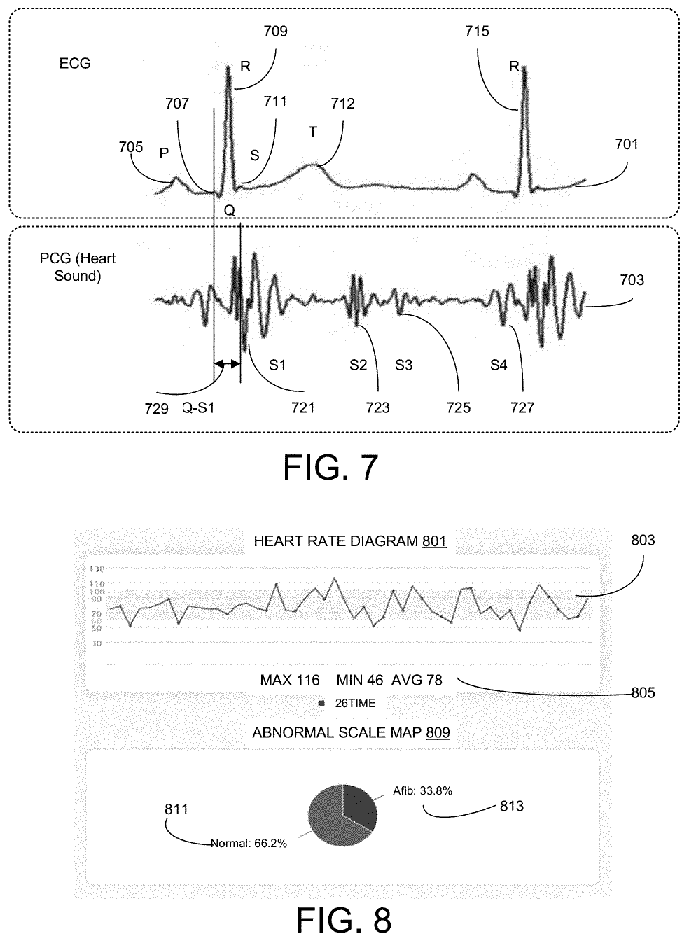

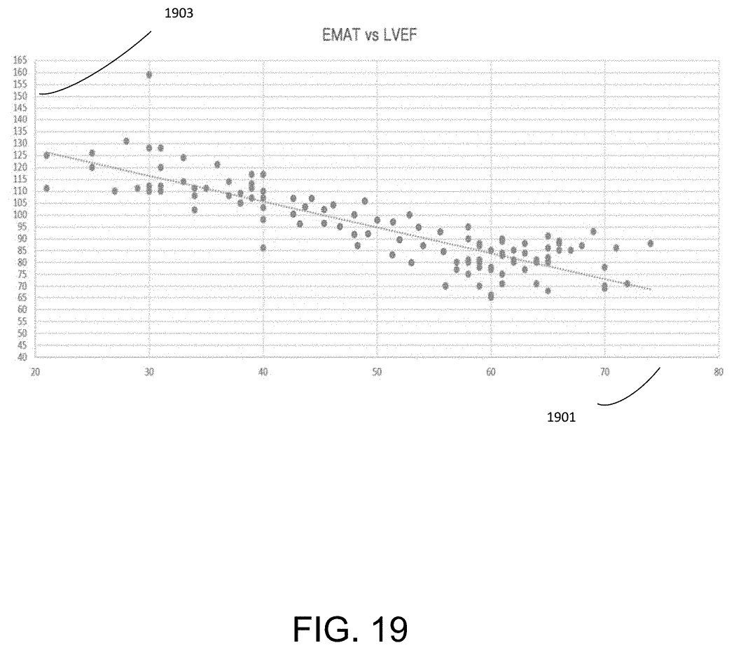

FIG. 7 illustrates exemplary diagrams of extracting EMAT parameters from the synchronized ECG (701) and PCG (703) signals. In ECG (701) signal, P (705), Q (707), R (709, 715), S (711), T (712) are standard parameters that can be extracted from the ECG (701) signal. In PCG (703) signal, S1 (721), S2 (723), S3 (725) and S4 (727) are standard parameters that can be extracted from the PCG (703) signals. For healthy people, the PCG signal will not have S3 and S4. For synchronized ECG and PCG, from ECG's Q to PCG's S1 is defined as electromechanical activation time (729) (EMAT). The left ventricular ejection fraction (LVEF), which is usually determined from color ultrasound imaging, is widely used as a measurement of the heart performance. From clinical experiments which is shown in FIG. 19, the x-axis is the LVEF obtained from color ultrasound imaging, the y-axis is the EMAT obtained from the synchronized ECG and PCG, the EMAT is determined to be negatively correlated to the left ventricular ejection fraction (LVEF). Therefore, the EMAT can also be used to measure the performance of the heart. The advantage of the wearable PCG and ECG is that the wearable device can collect synchronized PCG and ECG continuously so that the performance of the heart can be monitored continuously and in real time. The second heart sound (S2) in the PCG signal is produced by the closing of the atrioventricular valves and semilunar valves. It is closely related to the blood coming back to the heart driven by the blood pressure. After calibration, the digitized S2 can be used to monitor the average blood pressure continuously and in real time.

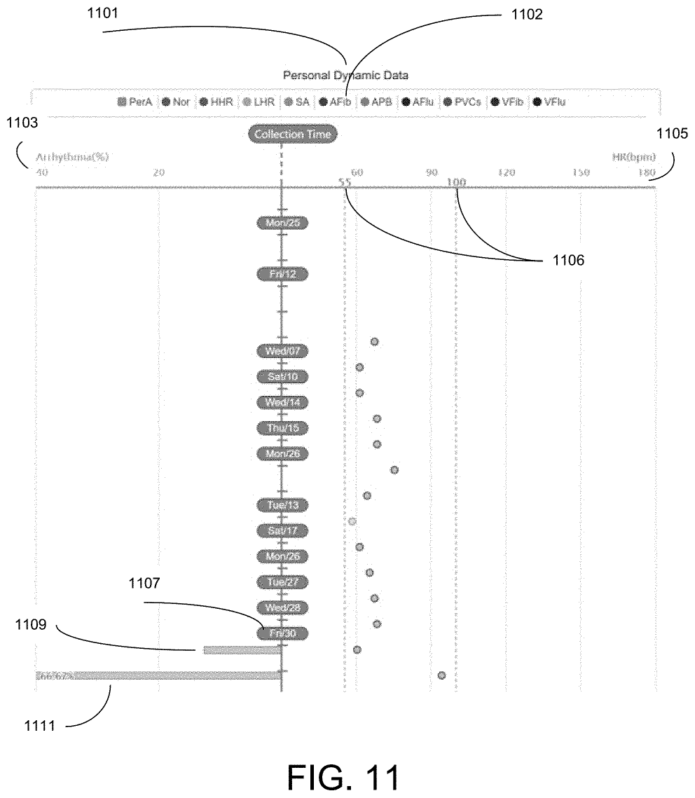

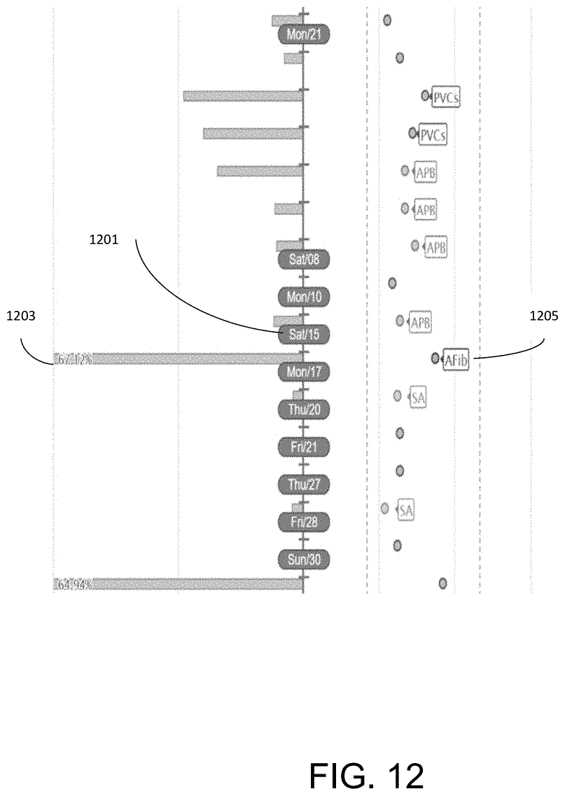

In one embodiment, one or more medical reports are generated by the synchronized multiple vital health measurements system. The medical report includes one or more medical reports based on multiple types of synchronized measurements obtained from multiple sensors. In one embodiment, a single report is generated based on a set of measurement results. The single report, as exampled in FIG. 8-FIG. 10, includes multiple records derived/generated based on measurements collected for a predefined single test period. In another embodiment, a multi-test report, as exampled in FIG. 11-FIG. 12 is generated with multiple tests over a period of report time. In one aspect, the wearable device with multiple sensors that can collect multiple vital health signals, digitize them, send them through the wireless network (such as Bluetooth, Wifi, 3G/4G/LTE/5G or other wireless networks) to a smart phone (or other mobile devices or a PC) or cloud-based data center. The smart phone (or other mobile devices or a PC) can display the data it received from the wearable device, store it at its local storage, and further relay the data to a cloud-based data center. When the cloud-based data center receives the data, it can automatically analyze and archive the data based on big-data mining, deep-learning algorithm, and various DSP algorithms. The analysis results will be crafted into a report. The results are then compared against a set of warning and alarm thresholds and if the results reach such thresholds, the proper warning and alarm will be part of the report. The analysis report will be sent back to the user and other people that the user has designated (such as healthcare professionals).

FIG. 8 is an example of the first part of a single test report automatically generated by the server or server-cluster side AI based analysis software. In one embodiment, the collected synchronized measurement results are digitized and analyzed using a deep learning software. For a given test, it has a heart rate diagram (801) which shows the heart rate changing curve (803) and the average heart rate (805). It also has an abnormal scale map (809) which shows the normal heart rate percentage (811) and abnormal heart rate percentage (813).

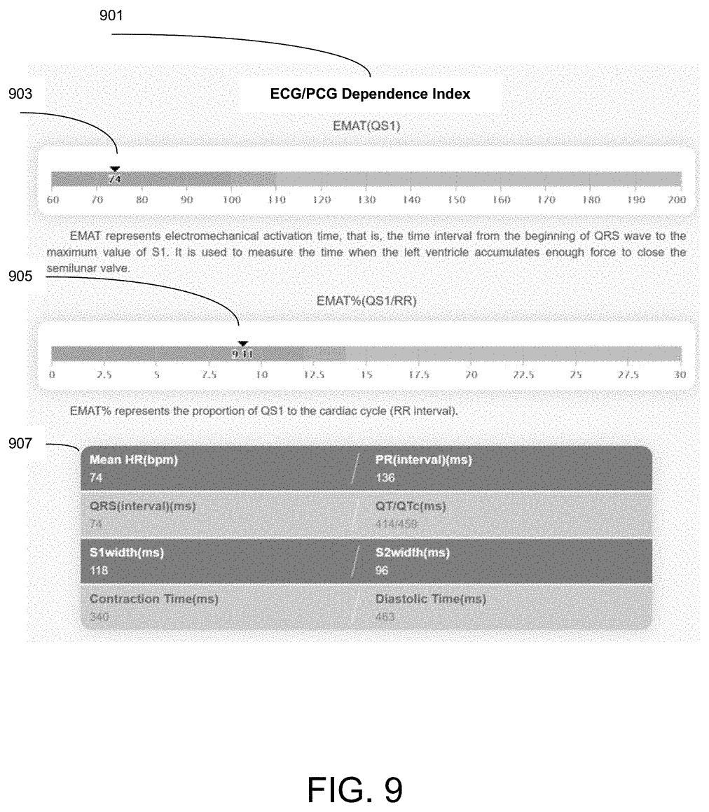

FIG. 9 is an example of the second part of a single test report automatically generated by the server or server-cluster side AI based analysis software. For a given test, it has an ECG/PCG dependence index (901) which lists EMAT (903), and EMAT % (905) in a color scaled meter. The larger these parameters are, the worse the heart performance is. Another set of parameters (such as mean HR, PR, S1 width, S2 width) are listed in another table (907).

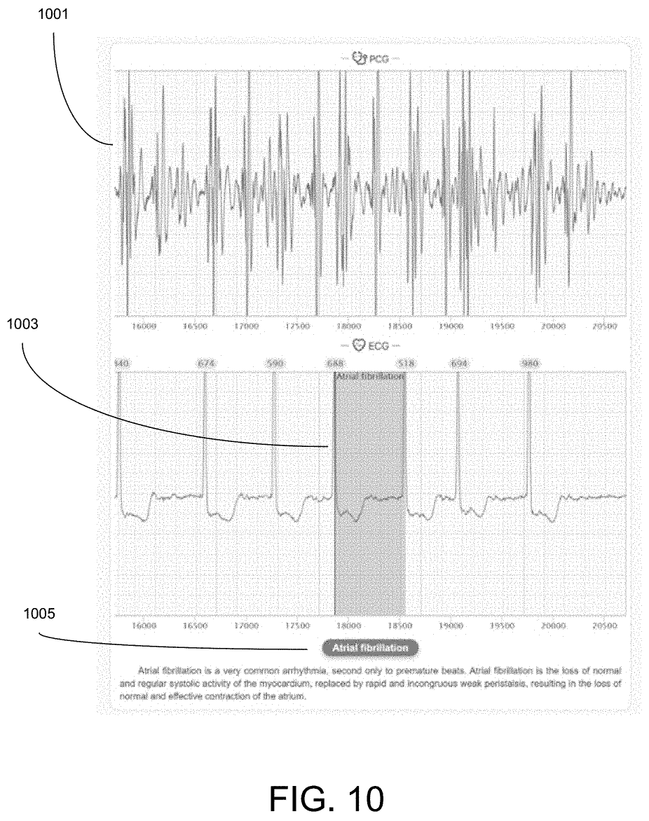

FIG. 10 is an example of the third part of a single test report automatically generated by the server or server-cluster side AI based analysis software. For a given test, it has shown the PCG waveform (1001) and ECG waveform and marked the problematic section (1003). It also gives the analysis result (1005) of the abnormal section in a special color.

FIG. 11 is an example of the first part of a test report for a period of time automatically generated by the server or server-cluster side AI based analysis software. It is called “Personal Dynamic Data” (1101). In the summary section, it will mark the problem for the test data (1102). As in this example, atrial fibrillation is detected in the test data. The left side is the percentage of arrythmia (1103). The right side is the heart rate range. Result-55 to result-100 are the normal ranges and marked with two vertical dash lines (1106). With every test case, the date of the test is marked (1107). If there is arrythmia, the percentage of arrythmia is shown (1109, 1111).

In one embodiment, one or more analysis algorithm is selected or preconfigured to perform data analysis on digitized measurement results. Different analysis algorithms include a wavelet, a short-time fast Fourier transformation (FFT), and a deep learning algorithm.

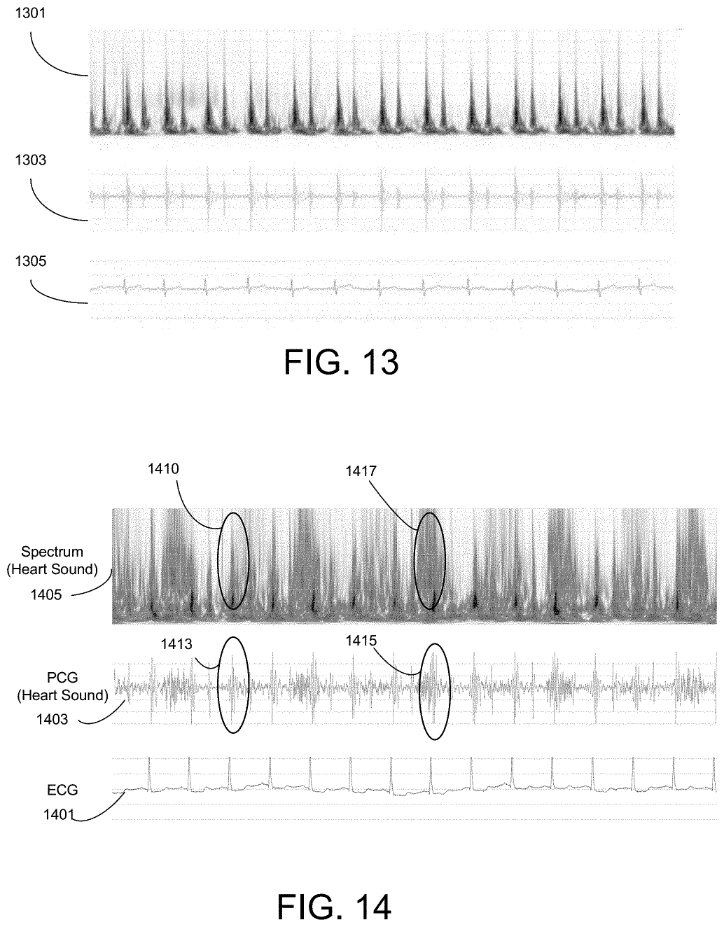

FIG. 13 is an example of using the wavelet transform to analyze the synchronized ECG and PCG signals. To get a better understanding of the PCG signal, time-frequency response analysis is a great help. Wavelet and short-time FFT can be employed. In this example, ECG (1305) and PCG (1303) must be sampled in synchronized mode. The wavelet time-frequency response is shown at the top line (1301).

FIG. 14 is an example of the synchronized ECG and PCG for a moderate COVID-19 patient. As we can see that the ECG (1401) of COVID-19 patient is normal. There is no arrhythmia. However, for the PCG (1403) signal, we can see the heavy respiration signals (1415) compared to the normal heart sound (1413). In the wavelet spectrum (1405), we can also see the heavy respiration energy (1417) compared to the normal heart sound spectrum (1410). For healthy people, cardiovascular patients, and even the COVID-19 patients with mild symptoms, the respiration signal within PCG is hard to be observed. However, for COVID-19 patients with moderate to severe symptoms, the respiration signal within PCG can be easily observed. During the COVID-19 crisis, many COVID-19 patients with mild symptoms had to stay at home. The wearable multiple vital health sensor can be used to monitor the synchronized ECG and PCG of the patient. Whenever the respiration signal is observed, the patient needs immediate medical care.

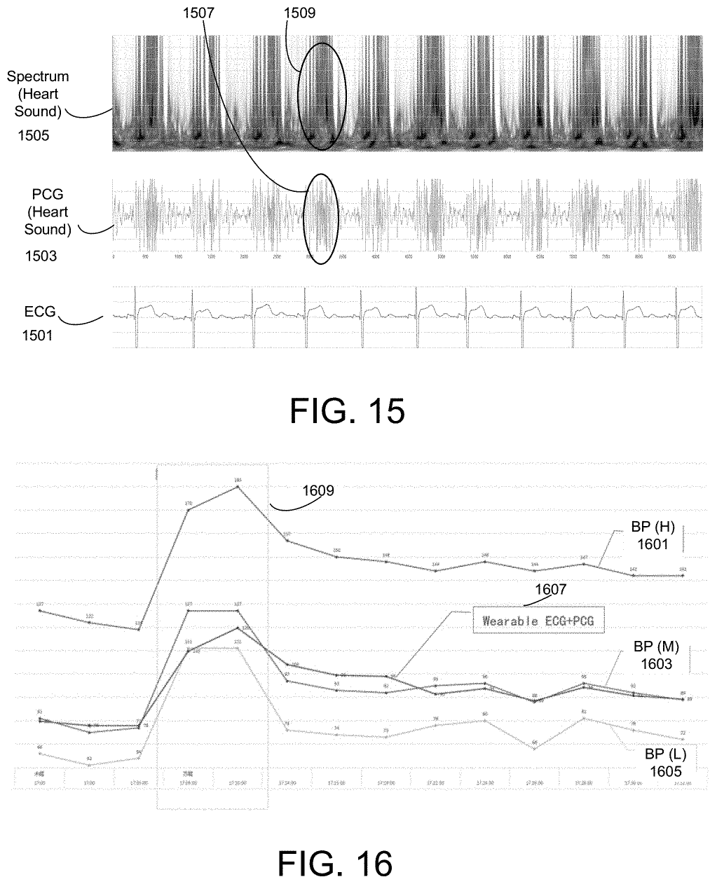

FIG. 15 is an example of the synchronized ECG and PCG for a child with congenial heart disease (CHD). As we can see that the ECG (1501) of the child with CHD is normal. There is no arrhythmia. However, for the PCG (1503) signal, we can see heavy noise (1507) between S1 and S2. As a matter of fact, we can hardly identify S1 and S2 (comparing the healthy people's PCG in FIG. 13). The heavy noise (1509) of PCG can also be observed on the wavelet spectrum (1505).

FIG. 16 is an example of using the synchronized ECG and PCG to monitor the blood pressure during the anesthesia procedure. The second heart sound (S2) in the PCG signal is produced by the closing of the atrioventricular valves and semilunar valves. It is closely related to the blood coming back to the heart driven by the blood pressure. After calibration, the digitized S2 can be used to continuously monitor the average blood pressure in real time. In this example, the systolic blood pressure (BPH) (1601) and the diastolic blood pressure (BPL) (1605) are obtained using the standard blood pressure measure instrument. The medium blood pressure (BPM) (1603) is obtained from the average of BPH and BPL. After calibration, the blood pressure estimated by the PCG is shown (1607), which is closely followed the BPM. As the patient wakes up from the anesthesia procedure, there is a jump in blood pressure (1609), and, gradually the pressure comes back to normal. During this period, the blood pressure estimated using the PCG (1607) reasonably matches the BPM (1603). Using the traditional non-invasive method to measure blood pressure can only be performed every 5 minutes, which is not preferred for continuous monitoring. For continuous blood pressure monitoring, the expensive invasive method has to be employed. By using the wearable synchronized ECG and PCG, we can achieve continuous non-invasive blood pressure monitoring.

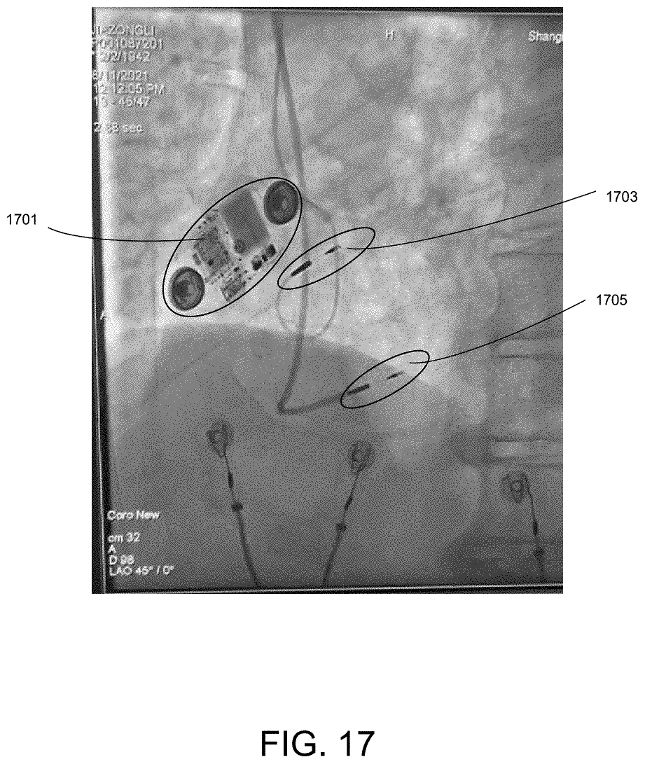

FIG. 17 is the X-ray image capture of using the wearable synchronized multiple vital health sensors during pacemaker implant operation. For the standard pacemaker implant operation, the main focus of the doctor is to place the electrodes of the pacemaker in a proper place so that the right pacing signals can be generated. Although much research has indicated that the placement and position of electrodes may also impact the heart performance of the patient, it is hard to adjust the electrodes for optimal heart performance. The main reason is that the usual method to evaluate heart performance is to use the color ultrasound imaging. It can only measure the heart performance at the given time. It cannot be done continuously. During the actual operation under an X-ray, performing even one ultrasound image is inconvenient, making performing multiple an unlikely possibility. Using the wearable synchronized ECG and PCG patch, we can continuously and non-invasively monitor the heart performance without any external wires, all the while occupying very little space. As shown in FIG. 17, the wearable sensor is patched onto the chest (1701). The two electrodes (1703, 1705) of the pacemaker can also be seen. During the operation, the doctor can adjust the place and position of the electrodes to not only get the perfect pacing signals but also minimize the impact on the heart performance.

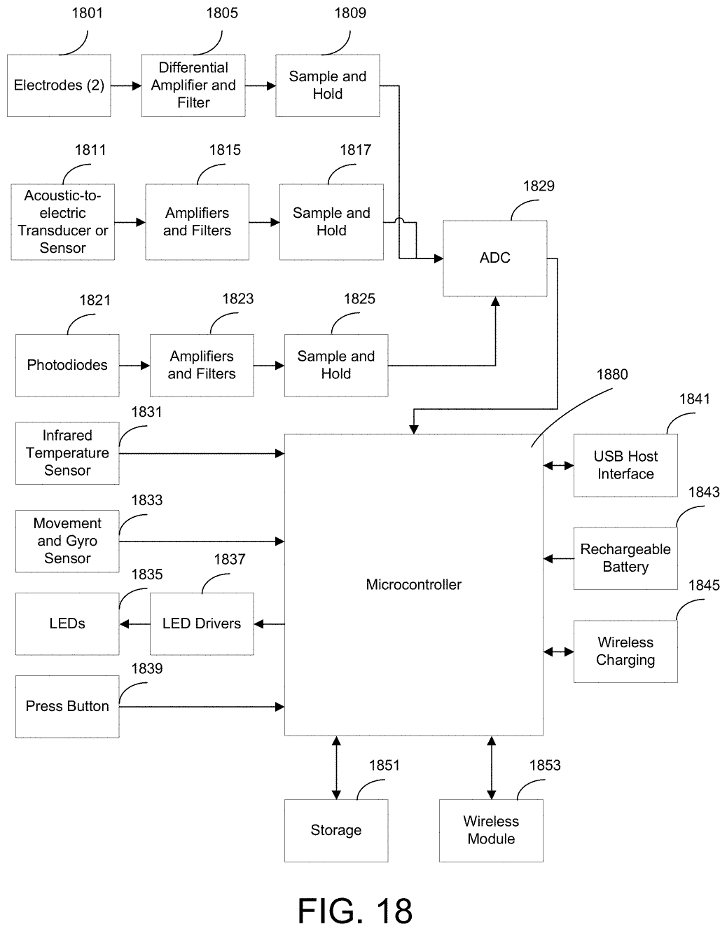

FIG. 18 has show the exemplary block diagram of the wearable multiple vital health sensors. It has an ultra-low power microcontroller (1880) as the central control unit. The microcontroller (1880) can have an optional USB interface (1841) so that the host can use it to fast download the data from internal storage (1851). It can also be used as a power supply to charge the re-chargeable battery (1343). For the model, which the user can wear during a shower, there will be a wireless charging module (1845) and charging wires to replace the USB interface (1341). The microcontroller (1880) has a wireless module (1853) to communicate with the smart device. Multiple wireless standards (such as Bluetooth, Wi-Fi, 3G/4G/LTE/5G, ZigBee, etc.) can be used. The microcontroller (1880) has internal storage (1851) so that the microcontroller can store part or all of the collected data. There is a multi-function press button (1839) and multiple LEDs (1835) driven by LED drivers (1837) connected to the microcontroller (1880). There is a motion, gyro, accelerator meter sensor (1833) connected to the microcontroller (1880), so the movements, orientation and acceleration of the wearable sensor can be monitored. A medical graded infrared temperature sensor (1831) is employed to detect body temperature. Most of microcontrollers have an internal temperature sensor. However, these temperatures are mainly used for monitoring the temperature of the microcontroller itself and the accuracy of these sensors cannot meet the 0.1-degree accuracy required by body temperature measurement. Therefore, a separated high precision infrared temperature sensor (1831) is employed. In order to detect the PPG, photodiodes (1821), amplifiers and filters (1823) and sample and hold circuit (1825) are used with LEDs (1835) and LED drivers (1837), along with ADC (1829) are employed to detect and digitize the PPG signal. In order to detect the sound, acoustic-to-electric transducer, or sensor (1811), amplifiers and filters (1815) and sample and hold circuit (1817), along with ADC (1829) are employed to detect and digitize the sound signal. In order to detect the ECG, two electrodes (1801), differential amplifier and filter (1805) and sample and hold circuit (1809), along with ADC (1829) are employed to detect and digitize ECG signal.

FIG. 19 illustrates an example diagram of the correlation between EMAT and LVEF. In one embodiment, blood pressure reports are generated continuously by performing a calibration, identifying, and digitizing S2 of continuously obtained PCG waveforms synchronized with an obtained ECG. As shown, the x-axis 1901 is the LVEF obtained from color ultrasound imaging; while the y-axis 1902 is the EMAT obtained from the synchronized ECG and PCG. From FIG. 19, we can see that there is close to an inverse linear relationship between EMAT and LVEF. To normalize the relationship, EMAT %, which is defined as EMAT divided by current heart rate or time between the current two ECG R peaks (as shown in FIG. 7, the time between 709 and 715) is used. Due to this relationship, from synchronized ECG and PCG, we can obtain EMAT and EMAT %, and they can be used to continuously predict the heart performance instead of using color ultrasound to obtain LVEF. FIG. 17 is an application of real time continuous EMAT and EMAT %. During the pacemaker operation, the doctors use EMAT and EMAT % to find the ideal place to place the pacemaker electrodes so that these electrodes can not only insert pacing signals correctly but also minimize the impact on heart performance. In one embodiment, the blood pressure is derived from the synchronized ECG and PCG waveform.

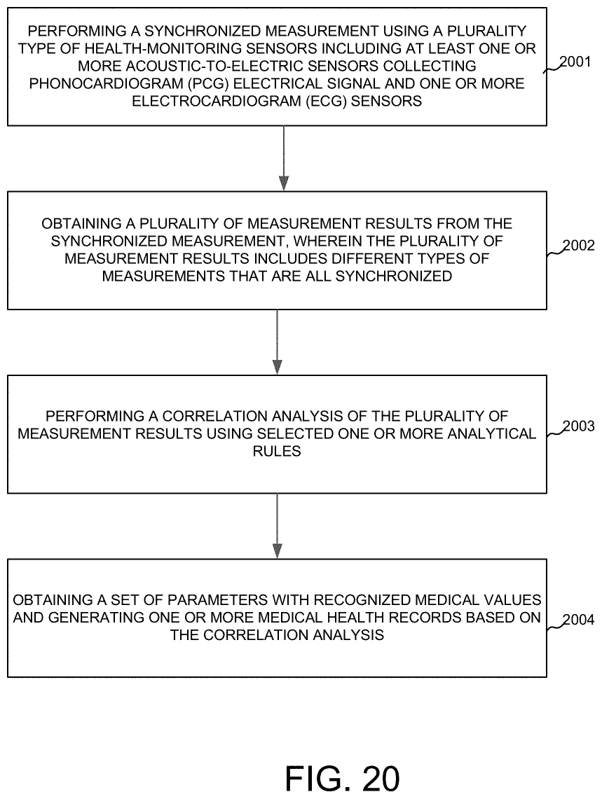

FIG. 20 illustrates an exemplary flow chart for the system to derive/generate medical records from synchronized multiple vital health measurements in accordance with embodiments of the current invention. At step 2001, the system performs a synchronized measurement using a plurality type of health-monitoring sensors including at least one or more acoustic-to-electric sensors collecting phonocardiogram (PCG) electrical signal and one or more electrocardiogram (ECG) sensors. At step 2002, the system obtains a plurality of measurement results from the synchronized measurement, wherein the plurality of measurement results includes different types of measurements that are all synchronized. At step 2003, the system performs a correlation analysis of the plurality of measurement results using selected one or more analytical rules. At step 2004, the system obtains a set of parameters with recognized medical values and generating one or more medical health records based on the correlation analysis.

Although the present invention has been described in connection with certain specific embodiments for instructional purposes, the present invention is not limited thereto. Accordingly, various modifications, adaptations, and combinations of various features of the described embodiments can be practiced without departing from the scope of the invention as set forth in the claims.

What is claimed is:

1. An apparatus comprising:
a wearable size center container that can be attached to a body;
a plurality of different types of sensors attached to the center container collecting a plurality sets of health signals, wherein the sensors include at least one or more acoustic-to-electric sensors collecting phonocardiogram (PCG) electrical signal and one or more electrocardiogram (ECG) sensors;
a control unit mounted in the center container, wherein the control module includes a synchronization circuitry that synchronizes measurements of the plurality of different types of sensors mounted on the center container; and
a wearable size patch with one or more electrodes, wherein each electrode is connected to the center container.

2. The apparatus of claim 1, wherein the different types of sensors further comprising: a photoplethysmography (PPG) sensor, a body temperature sensor, and an orientation and motion sensor.

3. The apparatus of claim 2, wherein one or more assistant sensors attached to the center container, comprising one or more environmental temperature sensor, and an environmental noise level sensor.

4. The apparatus of claim 1 further comprises: a configurable push button connected to the control module, wherein the push button is configured to perform a plurality of functions comprising an activation button, a panic button, and an event reminder button.

5. The apparatus of claim 1, wherein each electrode is connected to the center container with a button-sized metal connector.

6. The apparatus of claim 1, further comprising: a wireless communication circuitry, wherein the wireless communication circuitry communicates with one or more smart devices through a wireless network.

7. The apparatus of claim 6, wherein the apparatus is configured to monitor multiple vital health signals continuously and wirelessly.

8. The apparatus of claim 7, wherein the apparatus is used in one of different applications comprising in a telemedicine application, in a quarantined environment, within a hospital, and in an operation room.

9. The apparatus of claim 6, wherein the apparatus is attached to a user to continuously monitor a respiration sound caused by COVID-19 or other lung disease based on synchronized ECG and PCG obtained.

10. The apparatus of claim 6, wherein the apparatus is attached to a patient to continuously monitor a heart performance during a pacemaker operation, and wherein the heart performance is determined based on synchronized ECG and PCG obtained.

11. The apparatus of claim 1, further comprising:
one or more convertors connecting to the one or more micro acoustic-to-electric sensors and the array of voltage electrodes, wherein one or more convertors digitizes sensor waveform outputs;
a non-volatile storage that stores the digitized waveforms; and
a user interface unit that receives one or more user configurations for the apparatus.

12. A method comprising:
performing a synchronized measurement using a plurality type of health-monitoring sensors including at least one or more acoustic-to-electric sensors collecting phonocardiogram (PCG) electrical signal and one or more electrocardiogram (ECG) sensors;
obtaining a plurality of measurement results from the synchronized measurement, wherein the plurality of measurement results includes different types of measurements that are all synchronized;
performing a correlation analysis of the plurality of measurement results using selected one or more analytical rules; and
obtaining a set of parameters with recognized medical values and generating one or more medical health records based on the correlation analysis.

13. The method of claim 12, wherein the different types of sensors further comprising: a photoplethysmography (PPG) sensor, a body temperature sensor, and an orientation and motion sensor.

14. The method of claim 12, further comprising:
digitizing one or more waveforms obtained from corresponding sensors; and
analyzing digitized data with preconfigured an algorithm selecting from a wavelet, a short-time fast Fourier transformation (FFT), and a deep learning algorithm.

15. The method of claim 12, wherein the set of parameters with recognized medical values comprising electromechanical activation time (EMAT), EMAT percentage, a left ventricular ejection fraction (LVEF), and blood pressure.

16. The method of claim 15, wherein the EMAT is generated by correlating a set of synchronized ECG and PCG measurements obtained.

17. The method of claim 16, wherein the LVEF is obtained by negatively correlating the generated EMAT.

18. The method of claim 12, wherein the plurality of measurement results are obtained continuously and wirelessly from a wearable patch that collects the plurality of synchronized measurements.

19. The method of claim 18, wherein blood pressure reports are generated continuously by performing a calibration, identifying and digitizing S2 of continuously obtained PCG waveforms that are synchronized with an obtained ECG.

20. The method of claim 12, further comprising: identifying one or more problematic sections based on the correlation analysis.
